(19) United States
(12) Patent Application Publication
(10) Pub. No.: US 2023/0026220 A1
(43) Pub. Date: Jan. 26, 2023

(54) ORAL HEALTH ASSESSMENT FOR GENERATING A DENTAL TREATMENT PLAN

(71) Applicant: Adent ApS, Copenhagen K (DK)

(72) Inventors: Richard BUNDSGAARD, Copenhagen K (DK); Thøger BUNDSGAARD, Copenhagen K (DK)

(73) Assignee: Adent ApS, Copenhagen K (DK)

(21) Appl. No.: 17/910,714

(22) PCT Filed: Mar. 19, 2021
(86) PCT No.: PCT/EP2021/057072

§ 371 Date: Sep. 9, 2022

(30) Foreign Application Priority Data
Mar. 23, 2020 (DK) PA202070180

(51) Int. Cl. G16H 20/30; A61B 5/00; A61B 1/24; G16H 30/40

(52) U.S. Cl. CPC G16H 20/30; A61B 1/24; A61B 5/0077; A61B 5/6898; G16H 30/40; A61B 1/00009

(57) ABSTRACT

A computer program and computer-based system for remotely determining a dental treatment plan (10) of a user (30) of a personal computing device (20) by obtaining, using the personal computing device (20), at least one digital image (1) of the person’s oral cavity (31) and additional non-image data (2) comprising a choice (7) of a predefined topic (5) by said user (30). The digital images (1) and non-image data (2) are processed to extract dental health indicators (8) that are used to evaluate any dental condition (9) of said user (30). A dental treatment plan (10) is generated based on the dental condition (9) and a database (3) defining logical relationships between predefined topics (5) and actions (6) for said user (30) to take, wherein a selection of said actions (6) arranged in a regimen and displayed on a user interface (25) of said personal computing device (20).

### TECHNICAL FIELD

The present disclosure relates generally to the field of oral health, and more particularly to a computer-implemented method for assessing oral health and determining a dental treatment plan using digital images and non-image data (such as text input) obtained using a personal computing device.

### BACKGROUND

The identification of oral health issues - or the lack thereof -are crucial steps in combating oral illnesses or to supply preventive treatment for potential oral health problems. However, many people do not receive proper oral health assessment or have access to affordable dental plans, for several reasons. For example, they may live in rural areas far from a dental clinic and may, hence, not have access to dental health clinics, or they may not have the economic means to consult with a dental professional, let alone to pay for a potential diagnosis or treatment. Further, many people may be disinclined to spend time and money on regular dental checkups if they are not experiencing any apparent oral health problem, despite the fact that, sometimes, people may unknowingly have symptoms of compromised oral health or unknowingly be at risk of developing an oral health problem. In other cases, people may be experiencing a certain oral health problem or symptoms of an oral disease but may decide to wait before consulting with a professional, hoping that the problem or symptoms will go away. All these example scenarios are problematic since early and proper oral health assessment allows for early intervention and preventive treatment, thus preventing or reducing the occurrence or progress of many oral problems and diseases.

Systems for generating remote oral health hygiene plans, often as part of teledentistry, exist. These systems typically provide real-time and offline dental care such as diagnosis, treatment planning, consultation, and follow-up through electronic transmission of clinical data among patients and dental professionals, e.g. using smartphones or other mobile devices. Some known remote oral health assessment systems involve transmission of e.g. dental images from the patient. For example, the patient may use the front camera of his/her smartphone for capturing a dental image and may transmit the captured image via e.g. a web-based application on the smartphone for remote clinical assessment. However, these transmitted images are most commonly assessed visually by a dental professional, with or without taking into account additional clinical data regarding the patient’s clinical history from a local or remote database or their personal inputs. This process is not only costly but also requires a lot of time to get results, which may cause serious health issues in case a medical condition is recognized too late or if early prevention and mitigation plans are started too late. In similar fashion, early detection of oral health related medical conditions and providing actionable, easy-to-follow, personalized advice regarding such a condition can not only prevent the escalation of such conditions but save the patient or an insurance provider of the patient a lot of money otherwise spent on expensive treatments.

Accordingly, there is a need for technologies that can provide patients and dental professionals with fast, precise, remote dental plans based on the current state of the patient’s oral cavity and additional anamnestic information that the patients can provide using existing tools, such as a mobile device that is connected to the Internet and comprises an input interface (e.g. touchscreen) and a camera, without requiring medical expertise or training, to generate an easy-to-understand and affordable dental treatment plan for the user to follow.

There exists a further need for an efficient and reliable processing method which is capable of generating personalized dental treatment plans to prevent and/or improve dental conditions identified by processing both image and non-image input provided by an untrained user on a low-cost mobile device and which can provide, as a result, personalized dental treatment plan of dental conditions related to the oral health of the patient.

There exists a further need for systems and methods capable of showing such personalized dental treatment plan or assessments of dental conditions in a way that is customizable based on an end user such as a patient, dental professional or insurance provider, and can support a decision-making process for each end user individually.

### SUMMARY

It is an object to provide a method and corresponding computer program product and computer-based system that fulfills these needs and thereby overcomes or at least reduces the problems mentioned above. The foregoing and other objects are achieved by the features of the independent claims. Further implementation forms are apparent from the dependent claims, the description, and the figures.

According to a first aspect, there is provided a computer program comprising instructions which, when executed by a personal computing device, cause the personal computing device to:
access a database defining logical relationships between predefined topics and actions for the user to take, the database being arranged to be accessible by the personal computing device; obtain at least one digital image of the user’s oral cavity using a camera of the personal computing device;
obtain non-image data through the personal computing device, the non-image data comprising a choice of a predefined topic by the user;
extract dental health indicators from at least one of the at least one digital image and the non-image data; determining at least one dental condition of the user by evaluating the dental health indicators; and
generate a dental treatment plan comprising a selection of the actions arranged in a regimen, based on the choice of a predefined topic, the logical relationships between predefined topics and actions and the dental condition.

Providing such a computer program allows remotely detecting the current condition of a user’s oral cavity, by analyzing a combination of input visual and non-visual (anamnestic) information that the patients can intuitively provide using existing tools at hand, such as a low-cost mobile device that is connected to the Internet and comprises a simple input interface (e.g. a touchscreen) and a camera, without requiring medical expertise or training. Using identified dental features, the program can determine an instantly accessible dental plan, thereby providing cost-effective, personalized and quick remote dental assistance. The particular combination of image-based and non-image-based data input and analysis using a database defining logical relationships between predefined topics and actions for the user to take provides a more efficient and dynamically more adjustable dental regiment generation method than the prior art, resulting in a solution that is customizable through the database for an end user such as a patient, dental professional or insurance provider, and can support a decision-making process for each end user individually.

In a possible embodiment the dental treatment plan is a preventative plan, whereas in another possible embodiment the dental treatment plan is a treatment plan for pre-existing dental conditions.

In possible embodiments the determined dental condition can be the presence of a medical condition or symptom and/or the absence thereof.

In a possible implementation form of the first aspect generating a dental treatment plan comprises stochastic sampling using predefined rules on a weighted pool of actions. By using stochastic sampling, the initial plan generation is possible also when no user actions are recorded yet, in a simple and non-biased way, by “randomly” choosing from predefined actions.

In a further possible implementation form of the first aspect, generating a dental treatment plan (further) comprises Support Vector Machine (SVM) calculations using at least one of predefined rules, database entries (stated and revealed preferences), choice selections by the user, and determined dental conditions. This implementation makes possible to adapt and adjust dental treatment plans after an initial generation.

In a further possible implementation form of the first aspect, generating a dental treatment plan (further) comprises applying deep learning algorithms using at least one of predefined rules, database entries (stated and revealed preferences), choice selections by the user and determined dental conditions. This implementation makes possible to adapt and adjust dental treatment plans after an initial generation.

In a possible embodiment, the applied deep learning algorithms comprise a Long/Short Term Memory (LSTM) Recurrent Neural Network (RNN) that models “remembering and forgetting”.

In a further possible embodiment, the applied deep learning algorithms comprise Fully Connected Neural Network.

In a further possible embodiment, the applied deep learning algorithms comprise a Convolutional Neural Network that models “things that are near resemble each other” (such as actions that were perceived meaningful enough to complete today are also more likely to be perceived meaningful tomorrow than actions that were not perceived meaningful enough to complete today).

In further possible embodiments, generating a dental treatment plan (further) comprises applying statistical algorithms such as multi-factor regression, cluster analysis or the like.

In a further possible implementation form of the first aspect, generating a dental treatment plan (further) comprises applying a “best judgement” model of dental professionals, wherein a dental professional defines a set of actions, phases and milestones over a period of time for a user as an initial dental treatment plan, and the dental professional may further adjust actions, phases and milestones depending on the user’s clinical progression, stated preferences and revealed preferences during the treatment plan period. This type of model is strong for initialization of a dental treatment plan and to adapt a dental treatment plan while it is running.

In further possible embodiments, the regimen is a prescribed course of medical treatment, diet or exercise for the promotion or restoration of health.

In a possible embodiment, the at least one portion of the dental treatment plan is selected from a list of actions assigned for an upcoming period (e.g. part of a day, day, week etc.) according to the regimen.

In a possible implementation form of the first aspect the database comprises at least one filter matrix comprising matrix values defining weighted logical relationships between the predefined topics and actions, wherein each matrix value represents a relevancy of an action for a respective predefined topic determined based on personal preferences of a user or recommendations of at least one dental professional.

In a possible implementation form of the first aspect the at least one filter matrix comprises
at least one generic filter matrix comprising matrix values based on predefined rules and recommendations of dental professionals; and
at least one personal filter matrix comprising matrix values based on personal preferences of the user.

By providing at least one generic filter matrix and at least one personal filter matrix it is ensured that the dental treatment plan is personalized for a user to treat a pre-existing dental condition or to improve oral health in a standardized, easy-to-understand way.

In a possible implementation form of the first aspect the at least one generic filter matrix comprises
a topic-specific filter matrix defining logical relationships between topic-specific rules (such as “two of the same actions of the same type cannot occur on the same day”) and actions for the user to take, based on the choice of a predefined topic; and
a dental recommendation filter matrix comprising matrix values calculated as an average, based on recommendations of multiple dental professionals.

In a possible implementation form of the first aspect the at least one personal filter matrix comprises
a stated preferences filter matrix comprising matrix values based on stated personal preferences of the user, extracted from the non-image data; and
a predicted preferences filter matrix comprising matrix values based on predicted personal preferences of the user based on recorded user interactions between the user and the personal computing device (such as actions liked, rejected, completed, not completed) .

In a possible implementation form of the first aspect the database further comprises at least one social filter matrix comprising matrix values based on personal preferences of other users matched to the user based on similarity of user characteristics (such as gender, age, education level, history of dental plan compliance).

In a possible implementation form of the first aspect the non-image data comprises
plan-specific input comprising the choice of a predefined topic and at least one of an input relating to a devotion level and a plan duration; and
non plan-specific input comprising at least one of anamnestic information, and information regarding dental habits and past or current oral health symptoms;
wherein the non plan-specific input is used for extracting the dental health indicators, and
wherein the plan-specific input is used for generating the dental treatment plan by adjusting at least one of the selected actions, an intensity or a duration of the regimen.

In a possible implementation form of the first aspect obtaining the non-image data comprises performing a dialogue with the user using at least one of
text input through a user interface of the personal computing device,
selection input by selecting at least one of several presented answers on a user interface of the personal computing device, or
spoken input using an audio input interface of the personal computing device.

By providing various ways of input for the non-image data, users with e.g. hearing impairment or vision problems are also enabled to access accurate and easy-to-use dental treatment plans to improve their oral health or mitigate existing conditions which otherwise they might have difficulty accessing. It also provides the convenience of accessing dental recommendations in the comfort of their homes, thus also enabling persons with movement disabilities to access dental plans.

In a possible implementation form of the first aspect the actions are arranged in the regimen into action groups according to at least one of a time of the day (morning/evening/specific time of day) or a day of the week.

In a possible implementation form of the first aspect the actions for the user to take comprise at least one of
dental home care actions (such as brushing teeth or flossing),
habitual actions (such as drinking more water or avoiding sugary drinks or smoking),
tips and tricks for maintaining oral health, and
attending treatments at a dental professional;
and the personal computing device is configured to generate an alert for the user about upcoming actions (such as a visit to the dentist), based on current time data, according to the dental treatment plan.

In a possible implementation form of the first aspect obtaining the at least one digital image comprises prompting the user to take a photograph with the camera of the personal computing device of one or more areas of the oral cavity of the user; and wherein the obtained digital image is at least one of an intraoral or extraoral high resolution color photograph, preferably in the RGB or RGBA color space.

In a possible implementation form of the first aspect the computer program further comprises instructions which, when executed by a personal computing device, cause the personal computing device to: obtain follow-up data after displaying the dental treatment plan using the personal computing device, the follow-up data comprising at least one of
at least one additional digital image (such as an update of a previously obtained digital image or a series of images obtained for tracking and/or measuring progression of a dental condition),
additional non-image data (such as adjustment of duration or devotion, or change of topic),
recorded user interactions between the user and the personal computing device (such as actions liked, rejected, completed, not completed);
   update the logical relationships between predefined topics and actions in the database associated with any of the follow-up data; determine any new dental condition or a change of a dental condition based on any additional or changed dental health indicators extracted from the at least one additional digital image and the additional non-image data; and
   generate an adjusted dental treatment plan based on the additional non-image data, the logical relationships, and any new dental condition or change of a dental condition (e.g. by adjusting an intensity or a duration of the regimen).

In a possible further implementation form of the first aspect, the obtaining of the follow-up data may occur at specified time intervals or milestones.

In a possible implementation form of the first aspect the computer program further comprises instructions which, when executed by a personal computing device, cause the personal computing device to: record user interactions between the user and the personal computing device, the user interactions comprising at least one of a
like, rejection, completion, or non-completion of an action,
completion of a day of a dental treatment plan, and
receiving a score associated with a predefined event (such as achieving a predefined milestone of a dental treatment plan); and display a track record on the user interface, the track record comprising a visual summary of graphs, aggregated numbers, summary text or similar data aggregations of the user interactions.

By recording user interactions, it is possible to fine-tune the personalized recommendations to that of the user to be able to display dental plans that are not only accurate and easy-to-follow but are followable over a prolonged period of time to obtain a better oral health outcome.

In a possible implementation form of the first aspect the computer program further comprises instructions which, when executed by a personal computing device, cause the personal computing device to: obtain a review the generated dental treatment plan (e.g. by a dental professional) and apply any necessary adjustments according to predefined dental treatment guidelines.

In a possible implementation form of the first aspect the computer program further comprises instructions which, when executed by a personal computing device, cause the personal computing device to display at least one portion of the dental treatment plan, the adjusted dental treatment plan, or the reviewed dental treatment plan on a user interface of the personal computing device.

According to a second aspect, there is provided a system for determining a dental treatment plan of a user of a personal computing device, the system comprising:
a camera configured to obtain a digital image of an oral cavity of the user;
an input device configured to obtain non-image data comprising at least a choice of a predefined topic by the user;
database being arranged to be accessible by the personal computing device;
a computer-readable storage medium comprising a computer program according to any one of the possible implementation forms of the first aspect;
one or more processors operable to execute the computer program and perform operations according to any one of the possible implementation forms of the first aspect to generate a dental treatment plan; and
a user interface configured to display the dental treatment plan. By providing a system for determining a dental treatment plan, it is possible to obtain up-to-date information from the user and/or their healthcare professionals and to store and analyze the data obtained to provide and display a dental treatment plan.

In a possible implementation of the second aspect the system comprises:
a client device comprising the camera, the input device and the user interface; and
a server device in data connection with the client device, the server device comprising the database, the computer-readable storage medium and the one or more processors.

These and other aspects will be apparent from and the embodiment(s) described below.

### BRIEF DESCRIPTION OF THE DRAWINGS

In the following detailed portion of the present disclosure, the aspects, embodiments, and implementations will be explained in more detail with reference to the example embodiments shown in the drawings, in which:

FIG. 1 shows a flow diagram of a method for generating a dental treatment plan in accordance with the first aspect, using a system in accordance with the third aspect;

FIG. 2 illustrates displaying a portion of a dental treatment plan on a personal computing device in accordance with a further implementation form of the first aspect;

FIG. 3 shows a filter matrix in accordance with a further implementation form of the first aspect;

FIG. 4 shows a group of filter matrices used for generating a dental treatment plan in accordance with a possible implementation form of the first aspect;

FIG. 5 shows a flow diagram of a method for generating a dental treatment plan in accordance with a possible implementation form of the first aspect;

FIG. 6 illustrates displaying a portion of a dental treatment plan on a personal computing device in accordance with a further possible implementation form of the first aspect;

FIG. 7 shows a flow diagram of adjusting a dental treatment plan using follow-up data in accordance with a possible implementation form of the first aspect;

FIG. 8 illustrates a review process of a dental treatment plan by a dental professional in accordance with possible implementation forms of the first aspect;

FIG. 9 illustrates a displaying of a dental treatment plan on a user interface of a personal computing device in accordance with a further possible implementation form of the first aspect;

FIG. 10 is a block diagram of a system for determining a dental treatment plan for a user in accordance with a possible implementation form of the second aspect;

FIG. 11 illustrates possible steps of a dialogue for obtaining plan-specific input from a user in accordance with a possible implementation form of the first aspect; and

FIG. 12 illustrates possible steps of a dialogue for obtaining non plan-specific input from a user in accordance with a possible implementation form of the first aspect.

### DETAILED DESCRIPTION

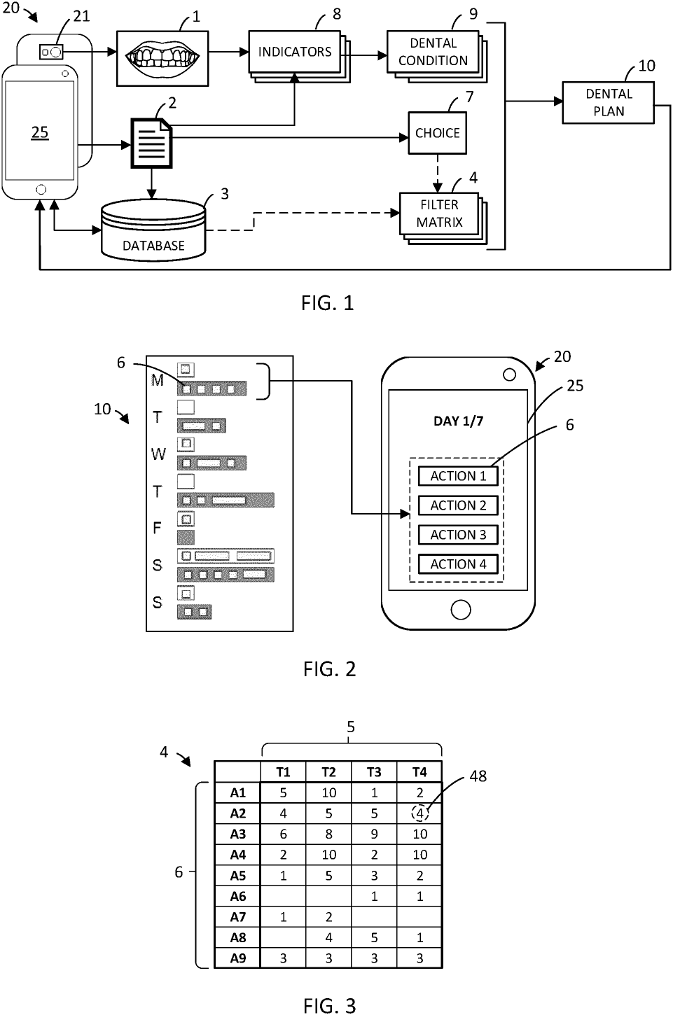

FIG. 1 shows a flow diagram of a method for determining a dental treatment plan 10 of a user 30 of a personal computing device 20 in accordance with the present disclosure, using a computer-based system such as for example the system shown on FIG. 10.

The user 30 may have observed symptoms of an oral health problem or disease and may wish to obtain digital images 1 of an area of his/her oral cavity 31 to have his/her oral health assessed based on the images 1 and additional non-image data 2. Or, the user 30 may not have observed any symptoms but may, nevertheless, wish to have his/her oral health assessed and be provided with a dental “treatment” plan even without any such symptoms.

In certain embodiments, the generation of a dental treatment plan 10 be part of an oral health treatment or a follow-up plan prescribed by a dental professional 32, or an initial or follow-up plan required by a health insurance provider, or specifically a dental insurance provider.

A database 3 is provided for defining logical relationships between predefined topics 5 and actions 6 for the user 30 to take, wherein the database 3 arranged to be accessible by the personal computing device 20, e.g. by arranging the database 3 directly on a storage medium of the personal computing device 20, or using a wired or wireless data connection.

Herein, predefined topics refer generally to focus areas regarding oral health a user may choose from, such as establishing a general dental wellness routine, treating an existing symptom or preventing an oral health condition (as will be listed below in detail). Further herein, actions refer to one-time or regular activities as well as pieces of information, reminders, eating or other habitual tips, or triggers for user interactions. The logical relationships between these entities can be established in any database format a computer can interpret, such as vectors or matrices, data tables and the like.

In an initial step, at least one digital image 1 of the oral cavity 31 of a user 30 is obtained using a camera 21 of a personal computing device 20. Herein, “oral cavity” may refer to e.g. lips, hard palate, soft palate, retromolar trigone (area behind the wisdom teeth), tongue, gingiva (gums), buccal mucosa, the floor of the mouth under the tongue, and/or teeth.

The user may capture the digital image(s) 1 with a digital camera of a mobile device or any suitable camera device (such as a small intraoral camera used by dental professionals) and/or may add existing images from a gallery available e.g. on or via a mobile device that were taken with a digital camera beforehand. The obtained digital image(s) 1 may be both an intraoral or extraoral high resolution color photograph(s), preferably in the RGB or RGBA color space.

For providing the at least one digital image 1, the user may be prompted to select a particular view/pose annotation describing the view/pose of the images, e.g. closed mouth view, bite view, bottom/lower arch view, upper arch view, bottom/lower lip view, upper lip view, closed bite anterior view, open bite anterior view, closed bite buccal view, open bite buccal view, roof of mouth view, floor of mouth view, side of mouth view, or frontal view, and may be guided to capture an image in the selected particular view/pose. In an embodiment the user may be required to provide at least one image for each of a set of predefined priority views, such as a `frontal view with closed bite', a bottom lip pulled down, exposing teeth in lower mouth and the inner bottom lip' and `top lip pulled up, exposing teeth in top mouth and the inner top lip'. In an embodiment, the user whose oral cavity is captured by the digital image 1 is the same person as the person operating the personal computing device 20 for obtaining the one or more digital images 1.In another embodiment, the user operating the personal computing device 20 is not the same person as the person whose oral cavity is captured on image. This may, for example, be the case when the person whose oral cavity is captured on image is a child or other person requiring help, e.g. from a parent or other family member, for capturing the one or more images of an area of his/her oral cavity.

In some embodiments, as will be explained below, digital images 1 can be provided not only for an initial assessment for generation of a dental treatment plan 10, but also during the actual building of a new/adjusted dental treatment plan 10 later on.

In addition to the above-described digital image 1, non-image data 2 associated with the same user is also obtained through the personal computing device 20. The non-image data 2 comprises at least a choice 7 of a predefined topic 5 by the user 30.

The non-image data 2 may further comprise anamnestic information about the user, wherein “anamnestic information” may refer to any type of information regarding the patient’s medical history including general health and symptoms or indications of comorbidities, as well as any current symptoms and habits or any further indications of the oral health of the user 30 (as will be described below in detail), preferably with a main focus on current dental habits 13B of the user 30.

Herein, the order of steps does not correspond to a strict order of execution - obtaining the non-image data 2 can happen in the same time, before and/or after obtaining the digital image(s) 1.

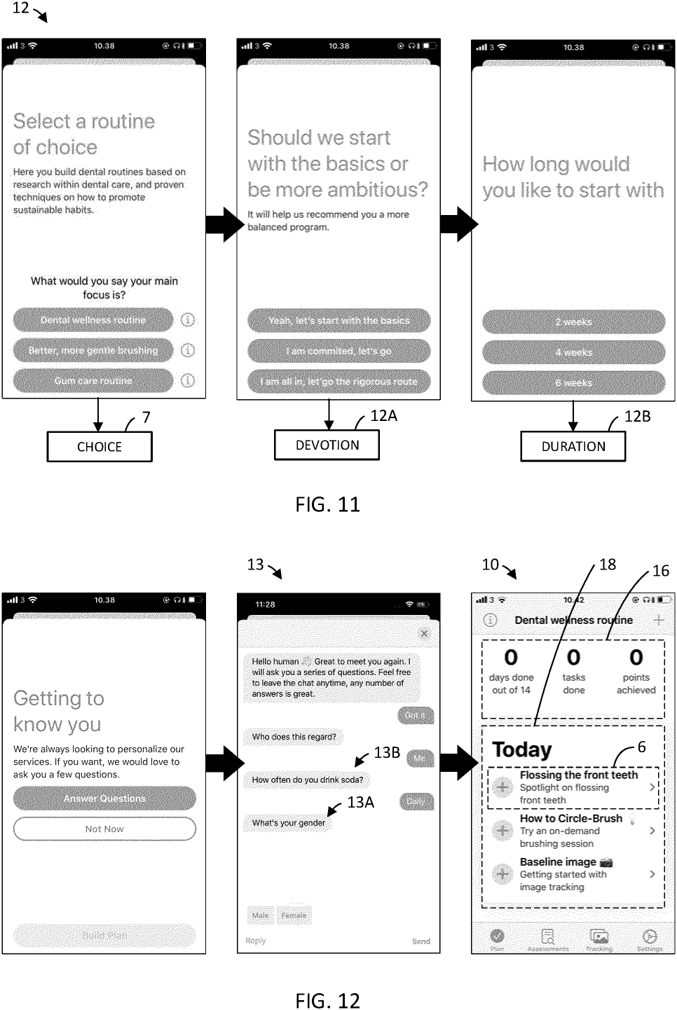

In an embodiment the non-image data 2 comprises self-reported user input given by the user 30 via e.g. a touchscreen interface of the personal computing device 20. In an embodiment the non-image data 2 may be obtained in the form of a dialogue, as illustrated throughout FIGS. 11-12, where the user 30 answers an automatically generated or predefined sequence of questions and the answers are recorded on the personal computing device 20.

In an embodiment, as illustrated also in FIG. 5, the questions may be received, and the answers may be given in the form of a text input 32, spoken input 34 or selecting input 33 from at least one of several presented answers in the form of a checklist, a slider bar, a visual representation, and/or free text. In an embodiment, a 3D representation of an oral cavity may be displayed to the user for indicating in the display screen an area corresponding to the area of the user’s own oral cavity associated with an oral health problem. The area may e.g. be a specific tooth. The user’s answers may be finite answers. For example, the user may select one or more suggestions from a checklist.

In an embodiment the sequence of questions to the user 30 may comprise questions relating to past and present lifestyle or behavioral data (such as tobacco use, diet, sugar intake, and oral hygiene habits such as brushing), known health or medical conditions (such as diabetes), symptoms, symptom triggers, and/or temporal contextual variables such as urgency. The questions may further relate to prepositions to oral risk, such as a user indicating a high mineralization of their spit, which in turn can lead to accelerated development of plaque/tartar.

In an embodiment the symptoms may be symptoms of at least one of gingivitis, periodontitis, dental caries, abrasion of tooth, bruxism, cold sore, erosion of teeth, fluorosis, herpes labialis, herpes zoster, or herpes infection.

In a preferred embodiment, the user 30 is presented with a text-based dialogue through a user interface 25 on a display of a personal computing device 20, the dialogue comprising a sequence of questions arranged to guide the user 30 through a process of combined input of both non-image data 2 and digital image(s) 1 in one flow.

Once obtained, both the non-image data 2 and the digital image(s) are processed to extract dental health indicators 8.

In an embodiment the at least one digital image 1 and the non-image data 2 are processed locally, e.g. on a mobile device, using at least one processor of the mobile device.

In another embodiment the at least one digital image 1 and the non-image data 2 are transmitted, using a computer network, to a server device 27 as shown in FIG. 10, and processed remotely on the server device 27.

In particular, the at least one digital image 1 may be processed using a statistical image segmentation algorithm trained to identify different visible segments from the digital image 1.The statistical image segmentation algorithm may be further trained to identify invisible segments based on the visible segments. Each identified segment may be linked to a tooth and its related area within the oral cavity or a non-tooth-related area within the oral cavity such as the gums, mucosa roof/floor of mouth, inner cheeks, tongue, or lips of a person.

In possible embodiments the oral cavity may be segmented into 200-1000 uniquely identifiable surface regions. These uniquely identifiable surface regions may represent a very granular surface identification, such as “the gum-attached lip-facing side of the bottom left incisor”.

In possible embodiments, the statistical image segmentation algorithm may use neural networks (e.g. MaskRCNN, YOLO, CNN) based on annotations using an annotation type of at least one of boxes, polygons, or masks. During training of these statistical image segmentation algorithms, digital images from different angles of the same oral cavity may be obtained (including images taken in one sitting or over time). This makes it possible to calculate which surfaces are typically/statistically invisible on which particular images, which may further inform the statistical algorithm trained to predict any invisible segments based on visible segments.

In a preferred embodiment, the statistical image segmentation algorithm uses landmark detection based on landmarks or focal points, supplemented with spatial or vector analyses of orthodontic features, such as the orientation of each tooth and the relation between adjacent and other teeth in the oral cavity. In some embodiments, the statistical algorithm trained to predict any invisible segments based on visible segments may be informed by the mapping of identified surfaces to predefined dental models, such as mapping onto a standard dental notation, e.g. a 5-surface tooth model or a 3-surface gum model.

The aforementioned statistical algorithms may further use a custom surface identification framework, such as clustering and/or distinguishing visually similar surfaces from each other, irrespectful of whether these are from a dental perspective nonrelated (or in the distinguishing case, that they are related) . An example includes that the “chewing surface” of molars typically go under the surface type “occlusal” in most dental settings, but visually this surface has some quite distinguishable features/sub-surfaces that are used in algorithms.

The identified segments may be further processed using a statistical object detection algorithm trained to identify dental health indicators 8 linked to the respective visible and/or invisible segments. Herein, the dental health indicators 8 may represent a wide range of oral health related findings such as fillings or missing teeth; presence of plaque; caries; pigmentation; color of the gums, lips or cheeks; presence of spit bubbles; etc. In this example, detected “spit bubbles” may indicate a “moist mouth” of a person which is the inverse of a “dry mouth”, the latter being a significant indication for developing dental conditions 9 such as cavities; whereas “presence of plaque” or “redness of the gums” may directly indicate different dental conditions 9.

In an embodiment the statistical object detection algorithm may use the non-image data 2 as additional input indicating what dental health indicators 8 to look for in the derivative segments of the digital image(s) 1.

In possible embodiments, the statistical object detection algorithm may use a neural network model, more preferably a convolutional neural network (CNN) model, based on annotations using an annotation type of at least one of boxes, polygons, or masks. In an embodiment the statistical object detection algorithm may use an R-CNN model (such as MaskRCNN).

In possible embodiments, dental health indicators 8 can be identified through a variety of different annotations, depending on the dental health indicator 8. Annotation types may include boundary boxes, polygons, focal points, and individual labelling of intelligently identified subset/local patches of the input image.

Once the dental health indicators 8 are extracted they are used for determining any dental condition 9 of the user 30. Herein, determining a dental condition 9 refers to an assessment based on a distillation or aggregation of extracted dental health indicators 8 using a rules-based engine or a predictive modelling engine, wherein a determined dental condition 9 may comprise at least one of an identified oral health problem, oral health disease, oral health risk, or a determination that no symptoms of any oral health problems or diseases are present in a respective area of the oral cavity.

In a possible embodiment, the predictive modelling engine analyzes patterns and correlations from the extracted dental health indicators 8, whereby dental health indicator markers may be established forming the basis for prediction of existing or future dental conditions 9. The predictive modelling engine may apply machine learning techniques including supervised and unsupervised learning algorithms to generate associations and correlations between dental health indicators 8 and dental conditions 9.

In possible further embodiments the dental condition 9 may include a diagnosis identifying a “medical finding” (which may refer to both normal and abnormal medical states), a referral to a dental professional, an estimate of the urgency of an oral health problem, a recommendation for self-treatment, etc. An abnormal medical state herein may refer to an oral disease or other oral health problem.

Further, herein, “oral health problem” or “oral disease” may refer to at least one of Abnormal taste in mouth, Abrasion of tooth, Acid reflux, Acute necrotizing ulcerative gingivitis, Addison’s disease, Alveolitis of jaw, Amalgam tattoo, Amelogenesis imperfecta, Anemia, Aphthous ulcer of mouth, Atrophy of tongue papillae, Black hairy tongue, Bleeding gums, Broken tooth injury, Bruxism (teeth grinding), Burning mouth syndrome, Cancer, Candidiasis, Cheek biting, Cheilosis, Chemical burn (mouth), Chicken pox (Varicella), Cold sore, Complete avulsion of tooth, Contusion, Crazing of enamel, Cyst, Dental caries, Dental filling lost, Dental peri-implant mucositis, Dental plaque, Dental restoration present, Dentinogenesis imperfecta, Denture stomatitis, Diastema of teeth, Early tooth exfoliation, Electrical burn, Enamel hypoplasia, Enlarged labial frenulum, Erosion of teeth, Eruption cyst, Erythema, Erythroleukoplakia of internal part of mouth, Excessive salivation, Fibroma, Fistula, Fluorosis, Fordyce’s disease, Fracture of tooth, Fractured dental restoration, Geographic tongue, Gingival recession, Gingivitis, Glossopyrosis, Hemangioma, Herpes labialis, Herpes zoster, Herpes infection, Hyperplasia of gingiva, Infectious mononucleosis, Leukemia, Leukoedema, Leukoplakia, Lichen planus (mouth), Linea alba of oral mucosa, Lip biting, Lipoma, Lymphadenopathy, Malignant melanoma, Malignant tumor of major salivary gland, Malocclusion of teeth, Measles, Melanin pigmentation (mouth), Melanocytic nevus (mouth), Melanoma in situ (mouth), Mucocele of mouth, Mumps, Necrosis of the pulp, Osteoporosis, Pain in throat, Papillary hyperplasia, Papilloma, Parulis, Pemphigoid, Pemphigus, Pericoronitis, Periodontal abscess, Periodontitis, Pseudomembranous thrush, Pulpitis, Pyogenic granuloma, Rubella, Sexually transmitted infectious disease, Sialolithiasis, Sinusitis, Smokeless tobacco keratoses, Smoker’s melanosis, Staining of tooth, Stomatitis, Subluxation of tooth, Syphilis, Teething syndrome, Temporomandibular joint disorder, Thermal burn, Tongue biting, Tonsillitis, Tooth absent, Tooth sensitivity to brush or floss, Tooth sensitivity to cold, Tooth sensitivity to palpation, Traumatic granuloma, Traumatic injury, Trigeminal neuralgia, Turner’s Tooth, Ulcer, Verrucous carcinoma, Vitamin deficiency, Wegener’s granulomatosis, White sponge nevus of mucosa, or Xerostomia. Further, herein, “oral health” or “oral health sign” may refer to at least one of Healthy gums, Healthy enamel, Healthy mucosa, Healthy tongue, Healthy lips, Healthy Roof of Mouth, Healthy Saliva Gland, or the absence of any oral health problem.

As an example, below are some dental health indicators 8 related to indications of a gum disease or inflammation as a dental condition 9:
Swelling of gums /type of indicator: form, contour, shape/,
Redness of gums /type of indicator: color, gradient, shade/,
Gradient of redness /type of indicator: color, gradient, shade/,
Bleeding gums,
Discolorations of teeth (due to lacking oral hygiene) /type of indicator: color, gradient, shade/,
External matter on teeth (due to lacking oral hygiene) /type of indicator: texture, surface, roughness/,
Presence of plaque,
Exposure of root /type of indicator: (primarily) color, gradient, shade,
Recession of gums /type of indicator: form, contour, shape/, and
Cliff separating enamel and dental /type of indicator: Form, contour, shape/.

A dental treatment plan 10 is then generated which comprises a selection of actions 6 arranged in a regimen, based on the choice 7 of a predefined topic 5 from the obtained non-image data 2, the logical relationships between predefined topics 5 and actions 6 defined in the database 3, and any above determined dental condition 9. Herein, “regimen” should be interpreted in a broader sense as a regular course of action(s), and in a preferred sense of a systematic plan designed to improve and/or maintain the health of a patient. In some embodiments the regimen may refer to a prescribed course of medical treatment, diet or exercise for the promotion or restoration of health of a patient.

Accordingly, in possible embodiments the dental treatment plan 10 is a preventative plan, whereas in another possible embodiments the dental treatment plan 10 is a treatment plan for pre-existing dental conditions. In preferred embodiments, as will be described below, the dental treatment plan 10 is configured to progress over time, with a primary focus on home care actions for a user 30 to take. It should be noted that the focus of the dental treatment plan 10 are not actions for dental professionals 32 to do in a dental clinic, and specifically not any type of surgery on a patient.

For the generation of the dental treatment plan 10 different methods and models can be used, based on e.g. what phase of the regimen the user 30 may actually be in, as will be explained below. An important aspect of all the applied methods is however that the user 30 by giving the above described non-image data 2 makes a choice 7 about what topic 5 (i.e. type of plan) to focus on, such as preventative measures against gingivitis/gums, or cavities, or dry mouth, and the like. The evaluation of the oral cavity 31 based on dental health indicators 8 and any determined dental condition 9 as described above then informs which type of actions 6 should be provided to the user 30, possibly in combination with the other inputs such as personal stated and/or revealed preferences from the database 3 (as will be explained below).

Generating the dental treatment plan 10 may comprise stochastic sampling using predefined rules (such as “two same actions 6 of same type or variants of same type cannot occur on the same day”) on a weighted pool of actions 6. By using such stochastic sampling, the initial generation of a dental treatment plan 10 becomes possible also when no actions of the user 30 are recorded yet, in a simple and non-biased way, by “randomly” choosing from predefined actions 6.

In other possible embodiments, generating a dental treatment plan 10 may comprise SVM calculations using at least one of predefined rules and entries in the database 3 (e.g. stated and revealed preferences), choices 7 by the user, and determined dental conditions 9. This implementation makes possible to adapt and adjust dental treatment plans 10 after an initial generation. In practice, first a set of actions 6 on day 1 of the regimen is displayed, which the user 30 completes or does not complete. Given the user’s 30 completion, explicitly stated preferences for types of actions 6, completion of other “similar” users 33 (based on same topic 5, same stated preferences, and same historical behavior at that stage of their plan 10) and other possible inputs, the Support Vector Machine algorithm calculates which set of actions 6 should be displayed to the user on Day 2. User 30 then completes or does not complete one or more actions 6 on Day 2, and the calculation/adjustment is repeated.

In other possible embodiments, generating a dental treatment plan 10 may (further) comprise applying deep learning algorithms, instead of SVM calculations, using the same kind of inputs of at least one of predefined rules and entries in the database 3 (e.g. stated and revealed preferences), choices 7 by the user, and determined dental conditions 9.

In a possible embodiment, the applied deep learning algorithms comprise a Long/Short Term Memory (LSTM) Recurrent Neural Network (RNN) that models “remembering and forgetting”.

In a further possible embodiment, the applied deep learning algorithms comprise Fully Connected Neural Network.

In a further possible embodiment, the applied deep learning algorithms comprise a Convolutional Neural Network that models “things that are near resemble each other” (such as actions 6 that were perceived by the user 30 meaningful enough to complete today are also more likely to be perceived meaningful tomorrow than actions 6 that were not perceived meaningful enough to complete today) .

In further possible embodiments, generating a dental treatment plan (further) comprises applying statistical algorithms such as multi-factor regression, cluster analysis or the like.

In a further possible embodiment, generating a dental treatment plan 10 comprises applying a “best judgement” model of dental professionals 32, wherein a dental professional 32 defines a set of actions 6, phases and milestones over a period of time for a user 30 as an initial dental treatment plan 10, and the dental professional 32 may further adjust actions 6, phases and milestones depending on the user’s 30 clinical progression, stated preferences and revealed preferences during the treatment plan period. This type of model is strong for initialization of a dental treatment plan 10 and to adapt a dental treatment plan 10 while it is running.

In an embodiment the dental treatment plan 10 can be generated locally on a personal computing device 20 acting as a client device 28, using at least one processor 24 of the personal computing device 20.

Similarly, in another possible embodiment the dental treatment plan 10 can also be generated on a remote server device 27 using the extracted dental health indicators 8 from the digital image(s) 1 and the non-image data 2, wherein the extracted data and further inputs (the choice 7 of a predefined topic 5 and the logical relationships between predefined topics 5 and actions 6) may be transmitted thereon using a computer network as shown in FIG. 10. The dental treatment plan 10 can then be transmitted for displaying to the user 30 on a user interface 25 of the personal computing device 20.

In some embodiments, the method or system may detect contradictory input from different sources such as different digital images 1 and non-image data 2. Each data source in such cases is weighted statistically with regard to the amount of information it carries. For example, a very blurry image will often carry low information, and a high-quality image with clearly visible findings will carry more. Similarly, it will often carry more information that the person is experiencing severe pain (indicated via obtained non-image data 2), even though the digital images 1 may also show that they might have healthy gum tissue. In fact, a person’s oral cavity will almost always show some degree of contradictory signs (e.g. strong enamel in some teeth, and progressed cavities in others or inflammation of the gums in the bottom part of the mouth, but non-inflamed in the top). Therefore, it is an integral part of the method and related system to handle and prioritize such contradictory signs.

The dental treatment plan 10 may highlight such detected or determined contradictory input for a dental professional 32 for further assessment and evaluation.

Finally, as illustrated in FIG. 2, at least one portion of the dental treatment plan 10 is displayed on a user interface 25 of the personal computing device 20 for the user 30. In a possible embodiment, the at least one portion of the dental treatment plan 10 is selected from a list of actions 6 assigned for an upcoming period (part of a day, day, week or the like) according to the regimen.

These actions for the user 30 to take may include:
dental home care actions such as brushing teeth or flossing,
habitual actions such as drinking more water or avoiding sugary drinks or smoking,
tips and tricks for maintaining oral health, and
attending treatments at a dental professional 32.

In possible embodiments the database 3 comprises at least one filter matrix 4 comprising matrix values 48 for defining weighted logical relationships between the predefined topics 5 and actions 6, wherein each matrix value 48 displays a relevancy of an action 6 for a respective predefined topic 5 determined based on personal preferences (be it stated or revealed) of a user 30 or recommendations of at least one dental professional 32.

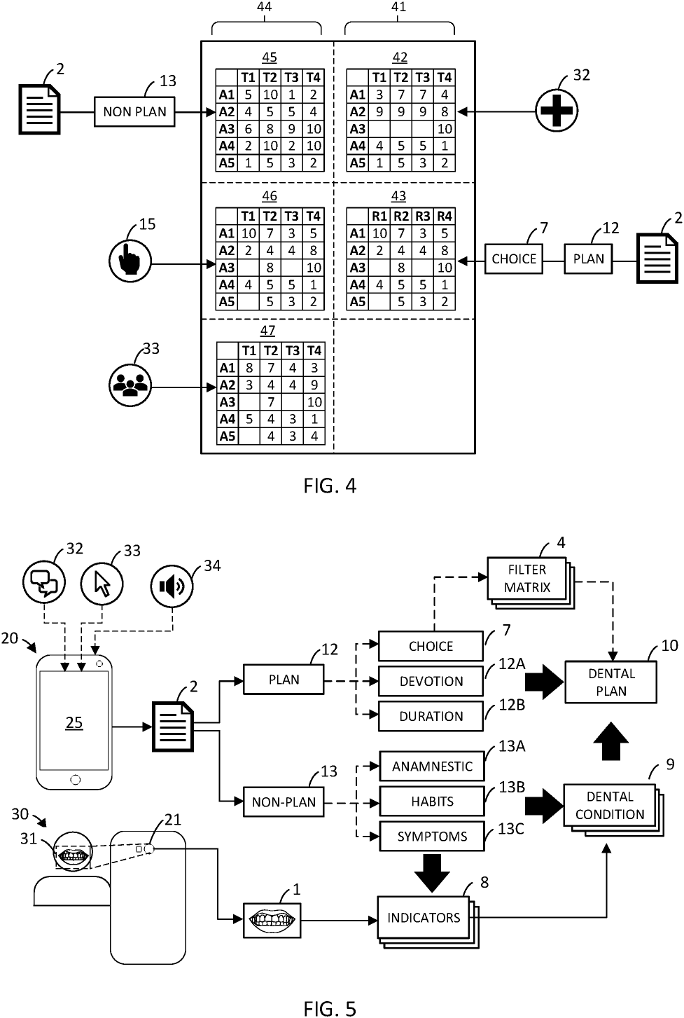

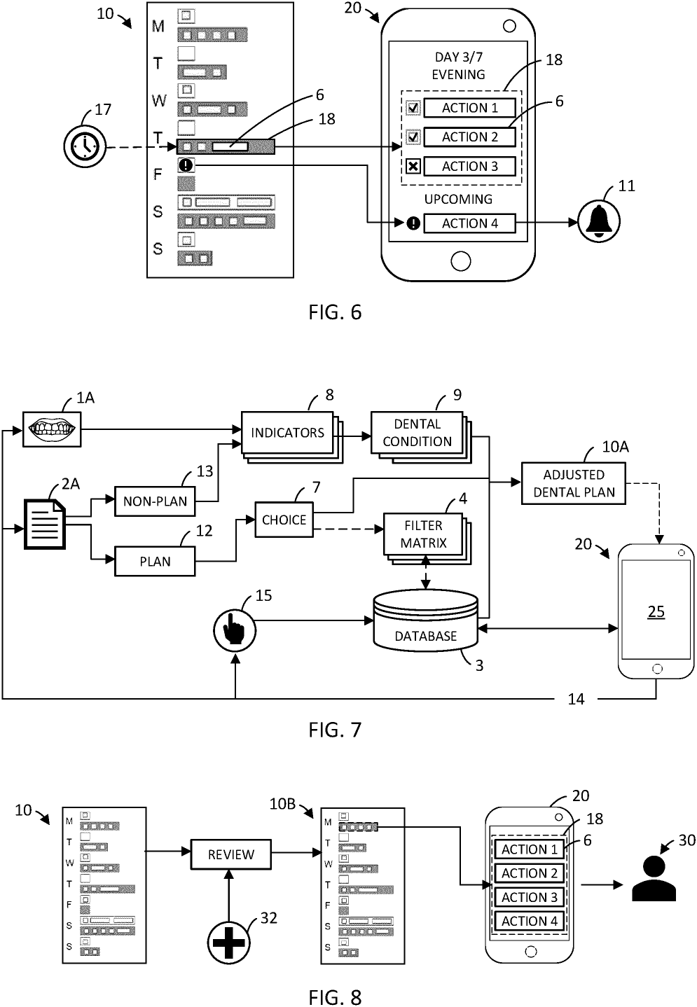

This aspect is illustrated in FIG. 3 showing exemplary matrix values 48, wherein rows illustrate possible actions 6 (A1-A9) for the user 30 to take and columns illustrate the topics 5 (T1-T4) that the user can choose. Values of the filter matrix 4 (e.g. column 2, row 5) may be given as a score from 0-10 of how relevant the action 6 is for the plan 10 from a dental professional’s 32 perspective. E.g. a 10 is “this is a very important action for the user to do if trying to fight emerging gingivitis”.

Note: which side is rows, and which side is columns is arbitrary. The matrix 4 can be flipped with no difference.

Examples for actions 6 may include:
Consult your dentist
Soft toothbrushing
Rigorous toothbrushing
How to Circle-Brush
Eat healthy
Sugary drinks
Flavored yoghurt
About dental crowns
About root canals
Smoking affects plaque
Avoid triggers for cold sores
Treating cold sores
Drink more water
Take a glass of water
Rinse daily with antiseptics
Acids only during meals
Take a piece of gum
Lessons on reflux
Take a pic
Hydrogen peroxide rinses

Examples for topics 5 the user 30 can choose from may include:
Dental wellness routine
Better, more gentle brushing
Emerging gingivitis
Gum care routine
Tracking root exposure
Emerging caries
Fighting erosion of teeth
Cold sores - Second or third occurrence
Removing dental calculus
Tracking Snus and its effects
Dental Crown: Getting started
Braces: Getting started
Braces: Correcting bad habits
Getting To Know Your Life with Braces
Removal of a wisdom tooth
Bad breath
Bleach
New Implants
Grinding teeth
Elder Care: Dentures
Following a child’s dental development as they grow
Growing up: The 6 Years Tooth
Parents’ Guide to Brushing with Their Kids
Full lips routine

FIG. 4 further illustrates an embodiment wherein the at least one filter matrix 4 comprises at least one generic filter matrix 41 comprising matrix values 48 based on predefined rules and recommendations of dental professionals 32; and at least one personal filter matrix 44 comprising matrix values 48 based on personal preferences of the user 30.

In the illustrated embodiment, the generic filter matrix 41 may comprise a topic-specific filter matrix 43 defining logical relationships between topic-specific rules (such as “two of the same actions of the same type cannot occur on the same day”) and actions 6 for the user 30 to take, based on the choice 7 of a predefined topic 5; and a dental recommendation filter matrix 42 comprising matrix values 48 calculated as an average, based on recommendations of multiple dental professionals 32.

In this embodiment, the personal filter matrix 44 may further comprise a stated preferences filter matrix 45 comprising matrix values 48 based on stated personal preferences of the user 30, extracted from the non-image data 2; and a predicted preferences filter matrix 46 comprising matrix values 48 based on predicted personal preferences of the user 30 based on recorded user interactions 15 between the user 30 and the personal computing device 20 such as actions 6 liked, rejected, completed, not completed.

In particular, stated personal preferences by the user 30 may include “Likes”, wherein users 30 explicitly state via the user interface 25 that they like specific actions 6, topics 5, phases or milestones. This may be done at relevant points in time (for actions 6 it can be done related to the actions 6 and for topics 5 it can be done at the end of a plan 10 or a prompted option at some key points of the plan 10. A “like” can either be in the form of e.g. “Like” (yes/no data), a 1-5 star rating, a 1-10 heart rating, “Share this” e.g. on Facebook (yes/no data), or similar ways.

The stated personal preferences by the user 30 may further include “Rejects”, wherein users 30 explicitly state via the user interface 25 that they reject specific actions 6, topics 5, phases or milestones. A “Reject” can either be in the form of “This is irrelevant for me”, “I don’t like this event”, or if user 30 deletes an action 6 or event in their “missed tasks” list and being asked “Do you want to avoid this type of event in the future?”, etc.

Completed actions 6 may be determined by the user 30 stating that they completed the action 6. This is recorded as a “revealed preference”.

Not completed actions 6, especially when recurring, are determined if a user 30 has been prompted to do an action 6 multiple times, but never does it - this is recorded as a “revealed preference”. Technically all non-completed tasks are a revealed preference, but often when just missing once or twice or sometimes, it is not a good indicator of preference.

In a possible embodiment, as also illustrated in FIG. 4, the database 3 may further comprise at least one social filter matrix 47 comprising matrix values 48 based on personal preferences of other users 33 matched to the user 30 based on similarity of user characteristics such as gender, age, education level, history of dental plan compliance.

FIG. 5 illustrates a further embodiment according to the present disclosure, wherein the non-image data 2 is obtained using the personal computing device 20 either as plan-specific input 12 and/or non plan-specific input 13. In this implementation, features that are the same or similar to corresponding features previously described or shown herein are denoted by the same reference numeral as previously used for simplicity.

The plan-specific input 12 may comprise a choice 7 of a predefined topic 5 and input relating to a devotion level 12A and a plan duration 12B, obtained via a set of questions. Examples of input that are plan-specific may be:
Intended focus of a plan (e.g. against gingivitis/gums);
Intended duration of a plan;
Intensity of a plan (e.g. “basic” vs “very ambitious” or “10 minutes per day” vs “1 minute per day”).

All inputs relating to a devotion level 12A may be transformed into a measure for the user 30 of “how much time and effort would you like to spend”. The inputs for devotion 12A can be grouped into initial adjustments and ongoing adjustments (received as additional non-image data 2A as will be described below).

Initial adjustments may include: “Start with the basics” (e.g. 3 minutes every 2nd day), “Ambitious” (e.g. 3 minutes every day), or “All in” (e.g. 10 minutes every day) , further customized by answers to questions such as “How often would you like prompts?” (e.g. every day, every second day), and “How many minutes would you want to engage?” (e.g. 1, 3, 5, 10, 20, 30).

Ongoing adjustments may include answers to mid-plan prompts like “Congratulations, you're level up! Would you like to increase your effort?” or adjust settings by the user 30 in personal preference page, e.g. as a reaction to the plan 10 being too ambitious/more ambitious than expected on initialization.

All inputs relating to a plan duration 12B may be transformed into a measure for the user 30 of “how long would you like your plan to be” (often with an ultimate unit of measure in days). Similarly as above, the inputs for plan duration 12B can be grouped into initial adjustments and ongoing adjustments (received as additional non-image data 2A as will be described below).

Initial adjustments may include input of weeks such as “2 weeks, 4 weeks, 6 weeks”, or definition of milestones, e.g. “Until next dental visit”.

Ongoing adjustments may include answers to mid-plan prompts similarly as above.

The non plan-specific input 13 in contrast may comprise anamnestic information, and information regarding dental habits and past or current oral health symptoms. Examples of input that are not plan-specific:
pregnancy of user 30;
gender of user 30;
how often user 30 brushes teeth;
how often user 30 flosses;
when user 30 last visited the dentist.

In this embodiment, as illustrated, the non plan-specific input 13 is used for extracting the dental health indicators 8, and the plan-specific input 12 is used for generating the dental treatment plan 10 by adjusting at least one of the selected actions 6 by the user’s 30 choice 7 directly or via the above described filter matrices 4, as well as the intensity or a duration of the regimen. As also illustrated, in possible embodiments obtaining the non-image data 2 comprises performing a dialogue with the user 30 using at least one of text input 32 through a user interface 25 of the personal computing device 20, selection input 33 by selecting at least one of several displayed answers on a user interface 25 of the personal computing device 20 as illustrated in FIGS. 11-12, or spoken input 34 using an audio input interface 29 of the personal computing device 20.

In an embodiment the non-image data 2 may be processed using a syntax analysis algorithm to extract a structured database of non-image signals. These extracted non-image signals may indicate at least one of:
behavioral data regarding oral habits, general health, medicine intake, comorbidities of periodontitis, history of trauma, history of oral procedures, pregnancy, or tobacco usage;
symptoms or signs of health regarding intraoral colors, surface structures, contours, wounds, texture, general health, taste in mouth, breath, saliva, healing process, pain; or
symptom triggers related to touch or icing, biting down, movement of tongue or jaw.

As also illustrated in FIG. 5, obtaining the at least one digital image 1 may comprise prompting the user 30 to take a photograph with the camera 21 of the personal computing device 20 of one or more areas of the oral cavity 31 of the user 30 for further processing as described before. The obtained digital image 1 may be an intraoral and/or extraoral high resolution color photograph, preferably in the RGB or RGBA color space.

FIG. 6 illustrates a further embodiment of the method according to the present disclosure, wherein the actions 6 are arranged in the regimen into action groups 18 according to at least one of a time of the day morning/evening/specific time of day or a day of the week to be displayed to the user 30 on the user interface 25 of the personal computing device 20. The personal computing device 20 may be further configured to generate an alert 11 (visual or sound alert or both) for the user 30 about upcoming actions 6 such as a visit to the dentist, based on current time data 17, according to the dental treatment plan 10. The user interface 25 may further be configured to provide feedback for the user about which actions 6 are completed and which are still to do according to the regimen.

FIG. 7 illustrates a further aspect of the method according to the present disclosure, wherein the dental treatment plan 10 can be adjusted and customized based on follow-up data 14 from the user 30 obtained after an initial generation of the dental treatment plan 10. In this implementation, features that are the same or similar to corresponding features previously described or shown herein are denoted by the same reference numeral as previously used for simplicity.

The follow-up data 14 for plan adjustment or customization may comprise:
at least one additional digital image 1A,
additional non-image data 2A, such as adjustment of duration or devotion, or change of topic 5, and
recorded user interactions 15 between the user 30 and the personal computing device 20, such as actions 6 liked, rejected, completed, not completed.

The obtained additional digital image 1A may comprise an update of a previously obtained digital image 1 or a series of images obtained for tracking and/or measuring progression of a dental condition 9 (such as increased or decreased inflammation of the gums) .

The obtaining of this follow-up data 14 may occur at specified time intervals (day, week, or month), phases or milestones defined in the regimen; or any time when the user 30 wishes to regenerate the dental treatment plan 10.

Examples for milestones may include:
Take a baseline of images and questionnaire;
Dental assessment; or
Visit to the dental clinic.

Examples for phases may include:
Antiseptic/Mouth rinse period;
Focused on extra flossing;
No solid food period (before surgery);
Coffee-only-in-the-morning for two weeks.

Once the follow-up data 14 is obtained, the logical relationships between predefined topics 5 and actions 6 in the database 3 (e.g. matrix values 48 of respective matrices as described above) associated with any of the follow-up data 14 can be updated; and any new dental condition 9 can be determined, or a change of a dental condition 9 may be tracked or measured based on any additional or changed dental health indicators 8 extracted from the at least one additional digital image 1A and the additional non-image data 2A.

After that, generating an adjusted dental treatment plan 10A is executed similarly as generating the dental treatment plan 10A as described above, based on the additional non-image data 2A, the logical relationships and the dental condition 9 (e.g. by adjusting an intensity or a duration of the regimen); and the adjusted dental treatment plan 10A can be displayed on the user interface 25 of the personal computing device 20.

FIG. 8 illustrates a further possible embodiment of the method according to the present disclosure, wherein the generated dental treatment plan 10 or adjusted dental treatment plan 10A is reviewed (e.g. by a dental professional 32) for applying any necessary adjustments according to predefined dental treatment guidelines, and then the reviewed dental treatment plan 10B is displayed on the user interface 25 of the personal computing device 20.

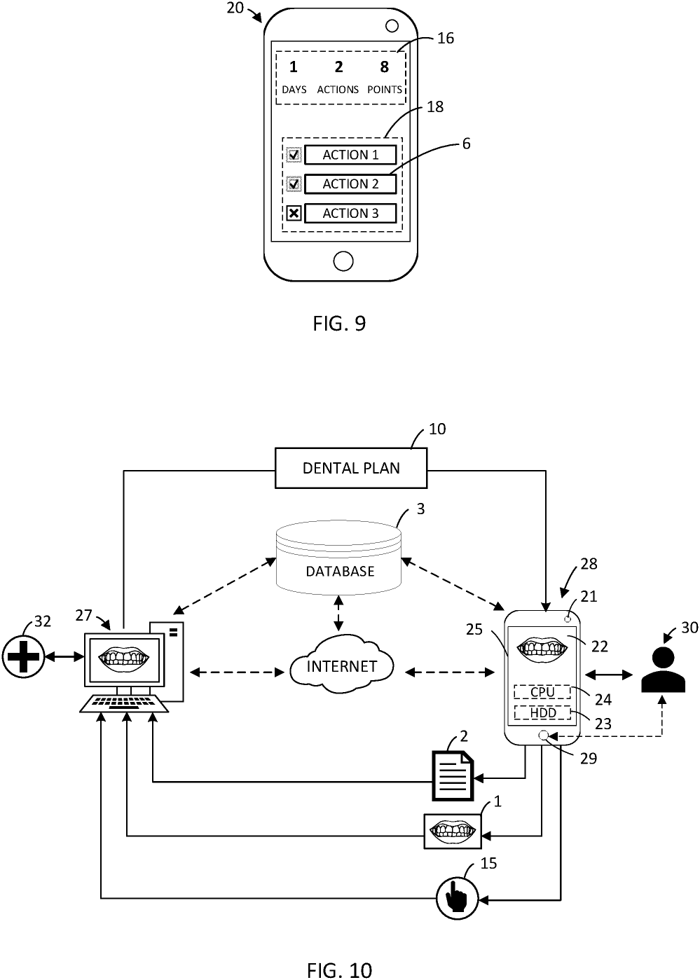

In a preferred embodiment, as illustrated in FIG. 9, user interactions 15 between the user 30 and the personal computing device 20 are recorded, which user interactions 15 may comprise:
like, rejection, completion, or non-completion of an action 6,
completion of a day of a dental treatment plan 10, and/or
receiving a score associated with a predefined event such as achieving a predefined milestone of a dental treatment plan 10.

These user interactions 15 may further include additional activities that go “beyond the plan” in the sense that users 30 might want to track weekly flossings, even if flossings are not a part of their dedicated plan 10.

These additional activities may include:
number of tooth brushing per day;
number of flossing per day;
number of use of interdental brush per day;
number of glasses of water per day;
number of mouth rinses per day;
number of tongue brushings per day.

After recording the user interactions 15, the method further includes displaying a track record 16 on the user 30 interface 25, the track record 16 comprising a visual summary of graphs, aggregated numbers, or similar data aggregations of the user interactions 15.

FIG. 10 illustrates a computer-based system for determining a dental treatment plan 10 of a user 30 of a personal computing device 20 in accordance with the present disclosure. In this implementation, features that are the same or similar to corresponding features previously described or shown herein are denoted by the same reference numeral as previously used for simplicity.

A client device 28 is provided, as part of the computer-based system, comprising at least one (digital) camera 21 configured to capture digital images 1 and means such as an input device 22 for obtaining further non-image input 2. The client device 28 may be the same as the personal computing device described before 20. The client device 28 may also be a portable computing device. In the embodiment illustrated in FIG. 10, the client device 28 is a smartphone that may comprise both a front camera above the display and a main camera on the rear side. In another embodiment, the client device 28 may be e.g. a tablet computer (tablet) with corresponding features (display and camera).

In an embodiment, the client device 28 may be configured to execute a software application (“app”) and comprises a digital camera 21 for capturing images 1 and a display for displaying the images 1 as part of a user interface 25, wherein the display and the digital camera 21 may be provided on opposite sides of the housing of the client device 28. In another embodiment, the camera 21 for capturing the digital images 1 may be a secondary camera provided on the same side of the housing of the client device 28 as the display. In an embodiment the display may comprise a touch screen that functions as the input device 22 and provides means for obtaining non-image data 2 from a user 30 by user interaction 15 with the help of a user interface 25. In an embodiment the client device 28 may further comprise an audio input interface 29 for obtaining spoken input 34 from a user 30.

The client device 28 may further comprise an integrated or external communications interface for connecting to other client devices directly or indirectly via a computer network. For example, the communications interface can include Wi-Fi enabling circuitry that permits wireless communication according to one of the 802.11 standards or a private network. Other wired or wireless protocol standards, such as Bluetooth, can be used in addition or instead. The client device 28 may further comprise an internal bus arranged to provide a data transfer path for transferring data to, from, or between the mentioned components of the client device 28.

FIG. 10 further illustrates a wireless system in accordance with the present disclosure, wherein the client device 28 may communicatively be connected to a network of computer nodes using an integrated or external communications interface, for transmitting image data and/or non-image data from the client device 28 to a server device 27 and/or for receiving data from a server device 27, such as a generated dental treatment plan 10 or input from a dental professional 32 received by the server device 27 using an input device, in a similar fashion as described above. Receiving such input from a dental professional 32 may happen after obtaining and transmitting image data and/or non-image data from the client device 28 to the server device 27, but may also happen before obtaining any input from the user 30, e.g. for generating an initial dental treatment plan 10 for the user 30.

The figure shows a diagrammatic representation of an exemplary wireless network, such as a cellular telephone network, the internet, or a cloud-based network, comprising the client device 28 and a server device 27 that can be e.g. a dental professional’s 32 workstation. Image data 1 and/or non-image data 2, as well as data from user interactions 15 may be transmitted from the client device 28 to the server device 27 for remote assessment, processing, and/or storage of the data. The transmission of data may be synchronous, asynchronous, or a mixture of synchronous and asynchronous data transmission.

As also illustrated in FIG. 10, a database 3 may also be implemented as part of the computer-based system to define logical relationships between predefined topics 5 and actions 6 for the user 30 to take. The database 3 may be stored on a computer-readable storage medium 23 of the server device 27, or on a computer-readable storage medium 23 of the client device 28, or even on a computer-readable storage medium 23 of a separate entity (e.g. a cloud-based storage provider) that is in data connection with at least one of the server device 27 or the client device 28 and can be called by a dental treatment plan application running on the server device 27 or the client device 28.

The method described above for determining a dental treatment plan 10 of a user 30 of a personal computing device 20 may at least partly be implemented as a computer program product encoded on a computer-readable storage medium 23 of a computer-based device, such as a server device 27 or client device 28 described above. The computer program product may in effect be realized in the form of a dental treatment plan application which may be executed by one or more processors 24 which may load the application software on a memory of a computer-based device, such as the server device 27 or client device 28 described above.

FIGS. 11 and 12 further illustrate examples of a dental treatment plan application for obtaining non-image data 2 and displaying a dental treatment plan 10 on a user interface 25.

In particular, FIG. 11 illustrates obtaining plan-specific input 12 through the personal computing device 20 using the user interface 25 as an input device 22 for a user 30 for selecting a choice 7 of a topic 5, then defining a level of devotion 12A and an intended duration 12B for the dental treatment plan 10 through selection input 33 by selecting at least one of several displayed answers.

FIG. 12 illustrates a separate flow obtaining non plan-specific input 13 through text input 32, and selection input 33 by selecting at least one of several displayed answers regarding anamnestic information 13A (including general health and symptoms or indications of comorbidities), information regarding dental habits 13B and past or current oral health symptoms 13C or any further indications of the oral health of the user 30, after which the dental treatment plan 10 is generated as described above, and actions 6 arranged in a regimen into action groups 18 according to at least one of a time of the day morning/evening or a day of the week are displayed on the user interface 25 alongside a track record 16 comprising a visual summary of graphs, aggregated numbers, or similar data aggregations of user interactions 15.

The various aspects and implementations have been described in conjunction with various embodiments herein. However, other variations to the disclosed embodiments can be understood and effected by those skilled in the art in practicing the claimed subject-matter, from a study of the drawings, the disclosure, and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measured cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems.

The reference signs used in the claims shall not be construed as limiting the scope.

1. A computer program embodied on a non-transitory computer-readable medium and comprising instructions which, when executed by a personal computing device (20), cause the personal computing device (20) to:
access a database (3) defining logical relationships between predefined topics (5) and actions (6) for the user (30) to take, the database (3) being arranged to be accessible by the personal computing device (20) and wherein the database comprises at least one filter matrix (4) comprising matrix values (48) defining weighted logical relationships between the predefined topics (5) and actions (6);
obtain at least one digital image (1) of the user (30)’s oral cavity (31) using a camera (21) of the personal computing device (20);
obtain non-image data (2) through the personal computing device (20), the non-image data (2) comprising a choice (7) of a predefined topic (5) by the user (30);
extract dental health indicators (8) from at least one of the at least one digital image (1) and the non-image data (2);
determine at least one dental condition (9) of the user (30) by evaluating the dental health indicators (8); and
generate a dental treatment plan (10) comprising a selection of the actions (6) arranged in a regimen, based on the choice (7) of a predefined topic (5), the logical relationships between predefined topics (5) and the weighted actions (6), and the dental condition (9).

2. A computer program according to claim 1, wherein the each of the matrix values(48) represents a relevancy of an action (6) for a respective predefined topic (5) determined based on personal preferences of a user (30) or recommendations of at least one dental professional (32).

3. A computer program according to claim 2, wherein the at least one filter matrix (4) comprises
at least one generic filter matrix (41) comprising matrix values (48) based on predefined rules and recommendations of dental professionals (32); and
at least one personal filter matrix (44) comprising matrix values (48) based on personal preferences of the user (30).

4. A computer program according to claim 3, wherein the at least one generic filter matrix (41) comprises
a topic-specific filter matrix (43) defining logical relationships between topic-specific rules and actions (6) for the user (30) to take, based on the choice (7) of a predefined topic (5); and
a dental recommendation filter matrix (42) comprising matrix values (48) calculated as an average, based on recommendations of multiple dental professionals (32).

5. A computer program according claim 3 , wherein the at least one personal filter matrix (44) comprises
a stated preferences filter matrix (45) comprising matrix values (48) based on stated personal preferences of the user (30), extracted from the non-image data (2); and
a predicted preferences filter matrix (46) comprising matrix values (48) based on predicted personal preferences of the user (30) based on recorded user interactions (15) between the user (30) and the personal computing device (20).

6. A computer program according to claim 1 , wherein the database (3) further comprises
at least one social filter matrix (47) comprising matrix values (48) based on personal preferences of other users (33) matched to the user (30) based on similarity of user characteristics.

7. A computer program according to claim 1 , wherein the non-image data (2) comprises
plan-specific input (12) comprising the choice (7) of a predefined topic (5) and at least one of an input relating to a level of devotion (12A) and a plan duration (12B); and
non plan-specific input (13) comprising at least one of anamnestic information (13A), and information regarding dental habits (13B) and past or current oral health symptoms (13C);
wherein the non plan-specific input (13) is used for extracting the dental health indicators (8), and
wherein the plan-specific input (12) is used for generating the dental treatment plan (10) by adjusting at least one of the selected actions (6), an intensity or a duration of the regimen.

8. (canceled)

9. (canceled)

10. Computer program according claim 1 , further comprising instructions which, when executed by a personal computing device (20), cause the personal computing device (20) to:
obtain follow-up data (14) after displaying the dental treatment plan (10) using the personal computing device (20), the follow-up data (14) comprising at least one of
at least one additional digital image (1A), such as an update of a previously obtained digital image (1) or a series of digital images (1) obtained for tracking or measuring progression of a dental condition (9),
additional non-image data (2A), such as adjustment of duration (12B) or devotion(12A), or choice (7) of topic (5),
recorded user interactions (15) between the user (30) and the personal computing device (20), such as actions (6) liked, rejected, completed, not completed;
update the logical relationships between predefined topics (5) and actions (6) in the database (3) associated with any of the follow-up data (14);
determine any new dental condition (9) or a change of a dental condition (9) based on any additional or changed dental health indicators (8) extracted from the at least one additional digital image (1A) and the additional non-image data (2A); and
generate an adjusted dental treatment plan (10A) based on the additional non-image data (2A), the logical relationships, and the new dental condition (9) or change of a dental condition (9).

11. Computer program according claim 1 , further comprising instructions which, when executed by a personal computing device (20), cause the personal computing device (20) to:
record user interactions (15) between the user (30) and the personal computing device (20), the user interactions (15) comprising at least one of a
like, rejection, completion, or non-completion of an action (6),
completion of a day of a dental treatment plan (10), and
receiving a score associated with a predefined event; and
display a track record (16) on the user (30) interface (25), the track record (16) comprising a visual summary of graphs, aggregated numbers, summary text or similar data aggregations of the user interactions (15).

12. (canceled)

13. A computer program according claim 1 comprising further instructions which, when executed by a personal computing device (20), cause the personal computing device (20) to display at least one portion of the dental treatment plan (10), the adjusted dental treatment plan (10A), or the reviewed dental treatment plan (10B) on a user interface (25) of the personal computing device (20).

14. A system for determining a dental treatment plan (10) of a user (30) of a client device (20), the system comprising:
a client device (28) comprising a camera (21), an input device (22), and a a user interface (25); and
a server device (27) in data connection with the client device (28), the server device (27) comprising a database (3), a computer-readable storage medium (23) and one or more processors (24); wherein
the camera (21) configured to obtain a digital image (1) of an oral cavity (31) of the user (30);
the input device (22) configured to obtain non-image data (2) comprising a choice (7) of a predefined topic (5) by the user (30);
the computer-readable storage medium (23) comprises a computer program according to claim 1 ;
the one or more processors (24) are operable to execute the computer program to generate a dental treatment plan (10); and
the user interface (25) is configured to display the dental treatment plan (10).

15. (canceled)
